(19) United States
(12) Patent Application Publication
(10) Pub. No.: US 2021/0277345 A1
(43) Pub. Date: Sep. 9, 2021

(54) CELL-CONTAINING CONTAINER, METHOD FOR EVALUATING TEST SUBSTANCE, AND METHOD FOR MANUFACTURING CELL-CONTAINING CONTAINER

(71) Applicants: Tomoyuki ARATANI, Kanagawa (JP); Daisuke TAKAGI, Ellicott city, MD (US); Hidekazu YAGINUMA, Kanagawa (JP); Waka LIN, Tokyo (JP); Shinnosuke KOSHIZUKA, Kanagawa (JP); Ryuya MASHIKO, Tokyo (JP); Tomoaki NAKAYAMA, Tokyo (JP); Atsushi MIYAOKA, Kanagawa (JP); Takeru SUZUKI, Saitama (JP); Minoru KO, Baltimore, MD (US)

(72) Inventors: Tomoyuki ARATANI, Kanagawa (JP); Daisuke TAKAGI, Ellicott city, MD (US); Hidekazu YAGINUMA, Kanagawa (JP); Waka LIN, Tokyo (JP); Shinnosuke KOSHIZUKA, Kanagawa (JP); Ryuya MASHIKO, Tokyo (JP); Tomoaki NAKAYAMA, Tokyo (JP); Atsushi MIYAOKA, Kanagawa (JP); Takeru SUZUKI, Saitama (JP); Minoru KO, Baltimore, MD (US)

(21) Appl. No.: 17/190,487

(22) Filed: Mar. 3, 2021

Related U.S. Application Data
(60) Provisional application No. 62/985,904, filed on Mar. 6, 2020.

(30) Foreign Application Priority Data
Feb. 5, 2021 (JP) 2021-017780

(51) Int. Cl. C12M 1/12; C12M 1/32; C12N 5/074

(52) U.S. Cl. CPC C12M 23/04; C12M 25/02; C12N 5/0607; C12M 23/12

(57) ABSTRACT

A cell-containing container includes at least one recessed part accommodating cells, in which the cells adhere to a bottom surface of the recessed part, and a cell density in a first region of the bottom surface is 250 to 20,000 cells/mm², and a cell density in a second region surrounding a periphery of the first region is less than 250 cells/mm².

### BACKGROUND OF THE INVENTION

### Field of the Invention

The present invention relates to a cell-containing container, a method for evaluating a test substance, and a method for manufacturing a cell-containing container. Priorities are claimed on U.S. Provisional Application No. 62/985,904, filed in the United States on Mar. 6, 2020, and Japanese Patent Application No. 2021-017780 filed in Japan on Feb. 5, 2021, the content of which are incorporated herein by reference.

### Description of Related Art

In recent years, in drug discovery development, interruption of development at a clinical trial stage has become a problem. This is due to animal species differences during a pharmacokinetic testing phase. Until now, in pharmacokinetic tests in a preclinical stage, the pharmacokinetics of drugs has been predicted using animals such as rats, dogs, and monkeys. However, it has become clear that the prediction is not practically valid in clinical trials using humans.

Furthermore, development of test methods that substitute for animal experiments is required from the viewpoint of the 3 R's of animal experiments (“Replacement,” “Reduction,” and “Refinement”).

Under these circumstances, studies are proceeding to test drugs in humans using human cells. As human cells, the following cells are used: primary cells, induced pluripotent stem (iPS) cells, embryonic stem (ES) cells, cells differentiated from iPS cells or ES cells, and the like. Since these cells are expensive, it is required to develop a technique for performing a reliable test with a smaller number of cells.

In drug tests using human samples, resin petri dishes, and plates such as 6-well, 12-well, 48-well, or 96-well plates are used as culture dishes. In general, these plates have about the same overall size, and the larger the number of wells, the smaller the size of one well. Each one well corresponds to one culture dish. Furthermore, 384-well plates with a smaller diameter and a larger number of culture dishes have begun to be used due to a recent trend toward miniaturization. A bottom part of these culture dishes is generally flat and plate-like, and a surface of the bottom part is used as a culture surface.

Conventionally, a method of performing comparison between patients by seeding living cells derived from a healthy subject and a patient in a culture dish, and performing an assay for comparing functions has been proposed. For example, Patent Document 1 discloses a cell culture kit including a microspace at a bottom part of a culture dish, in which living cells derived from a plurality of different donors are aggregated. Furthermore, Patent Document 2 discloses that mesenchymal stem cells were differentiation-induced into each of cells of the nervous system to perform comparison between a healthy subject and a patient.

### SUMMARY OF THE INVENTION

However, in a conventional cell-containing container, a cell adhesion region is the entire culture surface of the container. When cells adhere to the entire culture surface of the container, cell death may occur due to an increase in cell density at an edge portion of the culture surface. Furthermore, when cells adhere to the entire culture surface of the container, the cells may be dissociated from the culture surface due to tension generated when the cells proliferate confluently. As a result, the reliability of test results may decrease in some cases, especially in microscopic observation and optical analysis of fluorescence intensity.

An object of the present invention is to provide a cell-containing container capable of inhibiting cell death, cell dissociation, and the like caused by an increase in cell density.

The present invention provides a cell-containing container including at least one recessed part accommodating cells, in which the cells adhere to a bottom surface of the recessed part, and a cell density in a first region of the bottom surface is 250 to 20,000 cells/mm², and a cell density in a second region surrounding a periphery of the first region is less than 250 cells/mm².

According to the present invention, it is possible to provide a cell-containing container capable of inhibiting cell death, cell dissociation, and the like caused by an increase in cell density.

### BRIEF DESCRIPTION OF THE DRAWINGS

FIG. 1 is a schematic view illustrating a structure of a cell-containing container according to one embodiment.

FIG. 2A is a schematic view illustrating a structure of a cell-containing container according to another embodiment.

FIG. 2B is a schematic view illustrating a structure of a cell-containing container according to still another embodiment.

FIG. 3 is a schematic cross-sectional view illustrating a configuration of an inkjet head.

FIG. 4A is a fluorescence micrograph of cells in Experimental Example 1.

FIG. 4B is a fluorescence micrograph of cells in Experimental Example 1.

FIG. 4C is a fluorescence micrograph of cells in Experimental Example 1.

FIG. 4D is a fluorescence micrograph of cells in Experimental Example 1.

FIG. 5 is a graph showing a relationship between a cell density of iPS cells and efficiency of differentiation into nerve cells which are measured in Experimental Example 2.

FIG. 6A is a schematic cross-sectional view illustrating a frame member used in Experimental Example 3.

FIG. 6B is a schematic cross-sectional view illustrating the frame member used in Experimental Example 3.

FIG. 7A is a fluorescence micrograph of a cell cultured in Experimental Example 3.

FIG. 7B is a fluorescence micrograph of a cell cultured in Experimental Example 3.

FIG. 8A is a diagram illustrating a method for evaluating variation in cell adhesion in Experimental Example 4.

FIG. 8B is a diagram illustrating a method for evaluating variation in cell adhesion in Experimental Example 4.

FIG. 8C is a diagram illustrating a method for evaluating variation in cell adhesion in Experimental Example 4.

FIG. 9A is a fluorescence micrograph in Experimental Example 4.

FIG. 9B is a fluorescence micrograph in Experimental Example 4.

FIG. 9C is a Voronoi diagram showing a typical result of calculating a degree of aggregation of cells in Experimental Example 4.

FIG. 9D is a fluorescence micrograph in Experimental Example 4.

FIG. 9E is a fluorescence micrograph in Experimental Example 4.

FIG. 9F is a Voronoi diagram showing a typical result of calculating a degree of aggregation of cells in Experimental Example 4.

FIG. 10A is a graph showing a degree of aggregation of cells calculated in Experimental Example 4.

FIG. 10B is a fluorescence micrograph of cells cultured in Experimental Example 4.

FIG. 11A is a photomicrograph of a well of an MEA plate on which cells were seeded in Experimental Example 5.

FIG. 11B is a photomicrograph of a well of an MEA plate on which cells were seeded in Experimental Example 5.

FIG. 12A is a graph showing results of Experimental Example 6.

FIG. 12B is a graph showing results of Experimental Example 6.

FIG. 13 is a raster plot showing results of Experimental Example 7.

### DETAILED DESCRIPTION OF THE INVENTION

Hereinafter, embodiments of the present invention will be described in detail with reference to drawings in some cases. In the drawings, the same or corresponding parts are designated by the same or corresponding reference numerals, and duplicate description will be omitted. Dimensional ratios in each of the drawings are exaggerated for explanation and do not necessarily match actual dimensional ratios.

[Cell-Containing Container]

In one embodiment, the present invention provides a cell-containing container including at least one recessed part accommodating cells, in which the cells adhere to a bottom surface of the recessed part, the bottom surface has a first region and a second region surrounding a periphery of the first region, a cell density in the first region is 250 to 20,000 cells/mm², and a cell density in the second region is 250 cells/mm² or less.

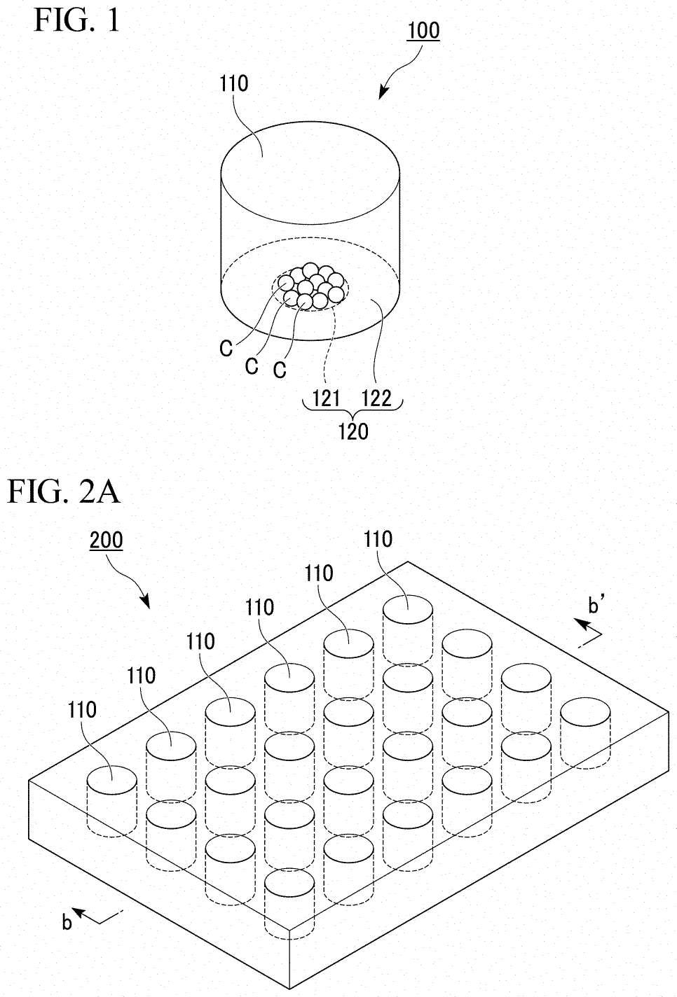

FIG. 1 is a schematic view illustrating a cell-containing container of the present embodiment. As shown in FIG. 1, a cell-containing container 100 includes a recessed part 110 accommodating cells C. The cells C adhere to a bottom surface 120 of the recessed part 110. The bottom surface 120 is a culture surface for cells, and has a first region 121 and a second region 122 surrounding a periphery of the first region 121. The second region 122 is in contact with an edge portion (end portion) of the bottom surface 120 of the recessed part 110. A density of the cells C in the first region 121 is 250 to 20,000 cells/mm², is preferably 500 to 10,000 cells/mm², and is more preferably 5,000 to 10,000 cells/mm² Furthermore, a density of the cells C in the second region is less than 250 cells/mm².

When a density of the cells C in the first region 121 is within the above range, for example, high efficiency of differentiation of iPS cells can be maintained in drug screening and the like. Furthermore, when a density of cells C in the first region 121 is within the above range, cell maturation proceeds. In a case of performing evaluation by image analysis, cells hardly overlap each other and evaluation can be efficiently performed by suppressing the number of cells to 500 to 1,000 cells/mm².

As will be described later in examples, according to the cell-containing container of the present embodiment, it is possible to reduce the influence of convection turbulent flow of a culture liquid generated during incubation in a cell culture environment by limiting a cell adhesion region to a region other than the edge portion (edge portion) of the culture surface. As a result, it is possible to inhibit cell death caused by an increase in cell density.

Furthermore, since a density of cells C in the second region is low, it is possible to inhibit the cells from being dissociated from the culture surface due to tension and the like generated when the cells proliferate confluently.

Furthermore, cells are present in a center portion (first region 121) of the culture surface at a cell density required for performing a highly reliable test. Therefore, a highly reliable test can be performed with a minimum number of cells. Accordingly, an amount of valuable cells used can be reduced. Furthermore, this is also advantageous in terms of cost.

The cell-containing container of the present embodiment may further include a sealing member that seals an opening of the recessed part. When the opening of the recessed part is sealed, the cell-containing container is easily transported. [0020]

In the cell-containing container of the present embodiment, the cells may be primary cells, an established cell line, iPS cells, cells differentiated from iPS cells, embryonic stem (ES) cells, or cells differentiated from ES cells. Furthermore, the primary cells may be differentiated cells or stem cells. Furthermore, the cells may be cells differentiated from the stem cells. The cells are preferably adherent cells. The cells are not limited to human-derived cells, and may be cells derived from mice, pigs, sheep, goats, cows, monkeys, and the like. Among the examples, the cells are preferably primary cells, stein cells, iPS cells, or cells differentiated from iPS cells, which are derived from a patient or a healthy subject.

Conventionally, animal cells were used for nerve cells, cardiomyocytes, and the like to perform tests in drug screening and the like because it is difficult to obtain those cells from humans. On the other hand, in recent years, since it has become possible to produce or obtain stem cells capable of differentiating into various cells such as ES cells and iPS cells, it has become possible to perform tests using human-derived cells. By differentiating stem cells derived from a patient with a specific disease, a part of the disease can be reproduced in vitro. Therefore, according to the cell-containing container containing such cells, it is possible to replace animal experiments and develop more accurate drug discovery.

The cell-containing container of the present embodiment includes a plurality of recessed parts 110. Each of the recessed parts 110 accommodates cells derived from the same patient or the same healthy subject, and the plurality of recessed parts 110 include recessed parts 110 each accommodating cells derived from different patients or different healthy subjects.

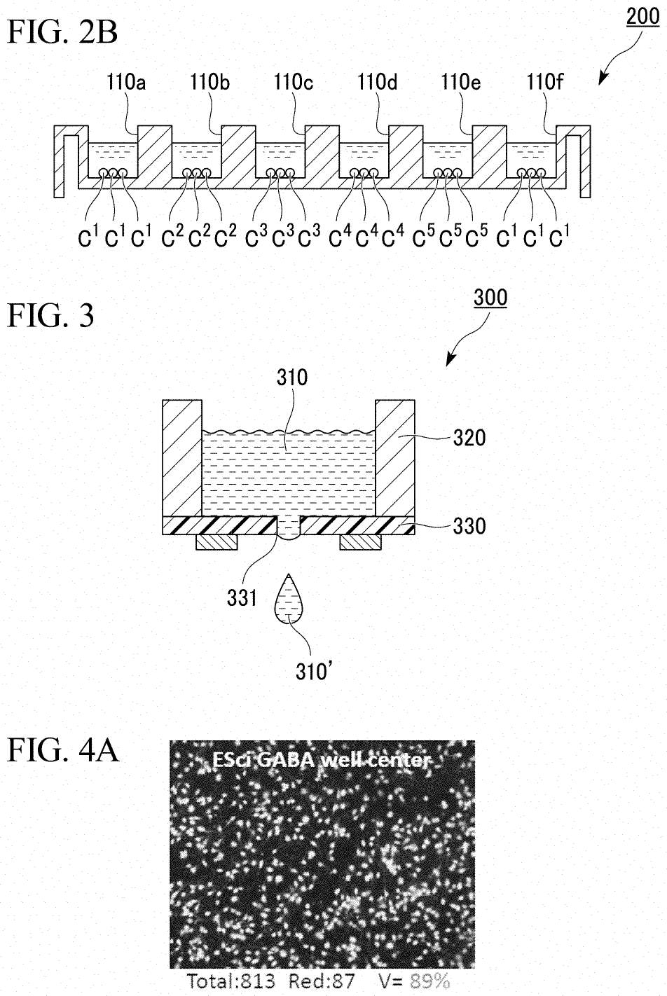

FIG. 2A is a perspective view illustrating a cell-containing container of the present embodiment. FIG. 2B is a cross-sectional view along a line b-b′ of FIG. 2A.

In an example of FIG. 2B, a cell-containing container 200 includes a plurality of recessed parts 110 (recessed parts 110a to 110f). In addition, cells accommodated in the recessed part 110 are derived from the same patient or the same healthy subject. That is, the origin of the cells accommodated in one recessed part 110 is the same. For example, the recessed part 110a and the recessed part 110f accommodate cells C¹ of the same origin. Furthermore, the recessed part 110b accommodates cells C² of the same origin. Furthermore, the recessed part 110c accommodates cells C³ of the same origin, and the same applies to the other recessed parts.

The plurality of recessed parts 110 may include recessed parts each accommodating cells derived from different patients or different healthy subjects. For example, the cells C¹ may be cells derived from a healthy subject A, the cells C² may be cells derived from a patient A, and the cells C³ may be cells derived from a patient B.

All cells accommodated in each of the recessed parts may be derived from different patients or different healthy subjects, or there may be a plurality of recessed parts accommodating cells of the same origin. In the example of FIG. 2B, the recessed part 110a and the recessed part 110f both accommodate the cells C¹ derived from the healthy subject A.

Using such a cell-containing container, it is possible to evaluate and compare the influences of drugs on healthy subjects and a plurality of patients.

In the cell-containing container of the present embodiment, a degree of aggregation of the cells in the first region which is calculated by Formula (1) is preferably 100% or less.

Degree of aggregation (%)=A/B×100 (1)

[In Formula (1), A indicates a standard deviation of area of Voronoi regions of each cells, and B indicates an average value of areas of Voronoi regions of each cells, where a Voronoi region of a cell is a region surrounded by perpendicular bisectors in a case where a perpendicular bisector is drawn between a nucleus of a cell and a nucleus of a cell adjacent to the cell].

The Voronoi region will be described later. As will be described later in the examples, when a degree of aggregation of cells is 100% or less, the cells are likely to be uniformly dispersed. Uniform distribution of cells is important for obtaining highly reliable test results with little variability. By controlling cell adhesion conditions and controlling a distance between cells at a single cell level, cells are inhibited from overlapping each other, and for example, it is possible to provide a cell-containing container that is optimal for evaluation using image analysis.

In the cell-containing container of the present embodiment, the recessed part may accommodate (i) nerve cells, and (ii) at least one cells selected from the group consisting of glial cells, vascular endothelial cells, and smooth muscle cells.

It is important to understand toxicity of drugs with respect to humans and responses of humans to drugs before clinical trials. Instead of culturing nerve cells alone, by co-culturing, with nerve cells in one recessed part, glial cells that activate an activity of nerve cells, vascular endothelial cells that nourish nerve cells, smooth muscle cells that maintain a complex structure of nerve cells, and the like, it is possible to perform an evaluation closer to an in vivo evaluation as compared to a case in which nerve cells are cultured alone.

Examples of nerve cells include GABAergic nerve cells, glutamatergic nerve cells, cholinergic nerve cells, monoaminergic nerve cells, histaminergic nerve cells, and the like. Which of these nerve cells a cell is can be determined by detecting the expression of a marker gene or marker protein specific to each cell, observing a morphology of a cell, detecting a function of a cell (spontaneous firing and the like), and the like.

Examples of glial cells include astrocytes, oligodendrocytes, microglia, and the like. Which of these glial cells a cell is can be determined by detecting the expression of a marker gene or marker protein specific to each cell, observing a morphology of a cell, and the like.

Furthermore, whether or not a cell is a vascular endothelial cell or a smooth muscle cell can also be determined by detecting the expression of a marker gene or marker protein specific to each cell, observing a morphology of a cell, and the like.

The cell-containing container accommodates nerve cells in the recessed part, and an electrode array may be provided on the bottom surface of the recessed part.

Examples of a cell culture container provided with an electrode array include a microelectrode array (MEA) plate and the like. The MEA plate is used for functional evaluation of nerve cells, cardiomyocytes, and the like. That is, the cell-containing container may be a cell-containing container in which cells are accommodated in an MEA plate.

(Recessed Part)

The recessed part is a compartment included in the cell-containing container and accommodates cells. In the cell-containing container of the present embodiment, the number of recessed parts is at least one, and is preferably 5 or more, and is more preferably 50 or more.

When the cell-containing container of the present embodiment includes a plurality of recessed parts, a distance between centers of two adjacent recessed parts is preferably 9.0 mm or less, is more preferably 5.0 mm or less, is even more preferably 4.5 mm or less, and is particularly preferably 2.25 mm or less. A center of a recessed part means a gravity center of a shape of an opening of the recessed part. Furthermore, a distance between centers of two adjacent recessed parts means a length of a line segment connecting the centers of the two adjacent recessed parts.

Examples of containers in which a distance between centers of two adjacent recessed parts is 9.0 mm or less include a multi-well plate, a micro-well slide, and the like. Examples of multi-well plates include well plates such as 96-well, 3M-well, and 1,536-well plates. Examples of micro-well slides include micro-well slides such as 192-well, 768-well, and 3,456-well micro-well slides.

A shape, a volume, a material, a color, and the like of the recessed part are not particularly limited, and the recessed part can be appropriately selected according to purposes.

A shape of the recessed part is not particularly limited as long as it can accommodate cells, and can be appropriately selected depending on purposes. Examples thereof include shapes of a flat bottom, a round bottom, a U bottom, a V bottom, and the like, among which a flat bottom is preferable.

A volume of the recessed part is not particularly limited and can be appropriately selected depending on intended purposes. For example, it may be 0.1 to 1,000 μL, may be 0.1 to 300 μL, may be 0.1 to 100 μL, or may be 0.1 to 10 μL, in consideration of an amount of a reagent used in a general evaluation method.

Regarding colors of the recessed part, the recessed part may be transparent, translucent, colored, or completely shaded. Furthermore, in a case of inspection with an optical system, a container in which a bottom surface portion is transparent and a side surface portion is colored is preferable from the viewpoint of inhibiting interference between adjacent recessed parts.

A material of the recessed part can be appropriately selected depending on purposes. For example, polyethylene terephthalate (PET), polystyrene (PS), polycarbonate (PC), triacetyl cellulose (TAC), polyimide (PI), nylon (Ny), low-density polyethylene (LDPE), medium density polyethylene (MDPE), acrylic materials such as vinyl chloride, vinylidene chloride, polyphenylene sulfide, polyether sulfone, polyethylene naphthalate, polypropylene, and urethane acrylate, organic materials such as cellulose and polydimethylsiloxane (PDMS), and inorganic materials such as glass and ceramics.

(Cell Adhesive Material)

A cell adhesive material may be disposed on the bottom surface of the recessed part. The cell adhesive material is not particularly limited and may be appropriately selected depending on purposes. Examples thereof include proteins selected from extracellular matrix, and the like.

Examples of proteins selected from extracellular matrix include fibronectin, laminin, tenascin, vitronectin, RGD (arginine-glycine-aspartic acid) sequence-containing peptide, YIGSR (tyrosine-isoleucine-glycine-serine-arginine) sequence-containing peptide, collagen, atelocollagen, gelatin, Matrigel (registered trademark), PuraMatrix, fibrin, mixtures thereof, and the like. A method of disposing the cell adhesive material on the bottom surface of the recessed part is not particularly limited. For example, a solution containing the cell adhesive material is injected into the recessed part, or a solution containing the cell adhesive material is jetted in the recessed part using an inkjet head or the like.

[Method for Evaluating Test Substance]

In another embodiment, the present invention provides a method for evaluating a test substance, the method including a step of adding a test substance into a recessed part of a cell-containing container which has at least one recessed part containing cells and in which a cell density in a center of a bottom surface of the recessed part is higher than that in an edge portion of the bottom surface of the recessed part; and a step of detecting spontaneous firing of nerve cells through an electrode array provided on the bottom surface of the recessed part.

It can also be said that the method for evaluating a test substance of the present embodiment is a drug screening method. The test substance is not particularly limited, and examples thereof include a natural compound library, a synthetic compound library, an existing drug library, a metabolite library, and the like. For example, a test substance that changes synchrony, frequency, and the like of spontaneous firing of nerve cells derived from a patient may be a candidate therapeutic agent for a disease.

In the evaluation method of the present embodiment, the cell-containing container may be the cell-containing container described above. That is, the cell-containing container may include at least one recessed part accommodating cells, in which the cell may adhere to a bottom surface of the recessed part, and a cell density in a first region of the bottom surface may be 250 to 1,500 cells/mm², and a cell density in a second region surrounding a periphery of the first region may be less than 250 cells/mm². Furthermore, cells, recessed parts, a material of cell-containing containers, and the like may be the same as those described above.

[Method for Manufacturing Cell-Containing Container]

In still another embodiment, the present invention provides a method for manufacturing a cell-containing container, the method including a step (jetting step) of controlling the number and positions of cells and jetting the cells by an inkjet technique, where the controlling is performed such that, in a recessed part of a container having at least one recessed part, a cell density in a center of a bottom surface of the recessed part is 250 to 20,000 cells/mm², and a cell density in an edge portion of the bottom surface of the recessed part is less than 250 cells/mm². The above-described cell-containing container can be manufactured by the manufacturing method of the present embodiment.

By controlling the number and positions of cells and jetting the cells by an inkjet technique, it is possible to manufacture a cell-containing container in which a cell density in a first region of a bottom surface of a recessed part is 250 to 20,000 cells/mm², and a cell density in a second region thereof is 250 cells/mm² or less.

A cell density in the first region is preferably 500 to 10,000 cells/mm², and is more preferably 5,000 to 10,000 cells/mm².

In the manufacturing method of the present embodiment, examples of containers include the same containers described above. Furthermore, examples of cells include the same cells described above.

In the jetting step, cells are jetted by jetting a cell suspension as liquid droplets by an inkjet technique. In the present specification, a liquid droplet means a mass of liquid that is collected by surface tension. Furthermore, jetting means that a cell suspension is sprayed as liquid droplets.

The jetting step is preferably performed by an inkjet head including at least a liquid-holding part which holds a cell suspension containing cells, a film-like member on which a nozzle is formed and which jets, from the nozzle, the cell suspension held in the liquid-holding part as liquid droplets by vibration, and an atmospheric opening part which opens an inside of the liquid-holding part to the atmosphere.

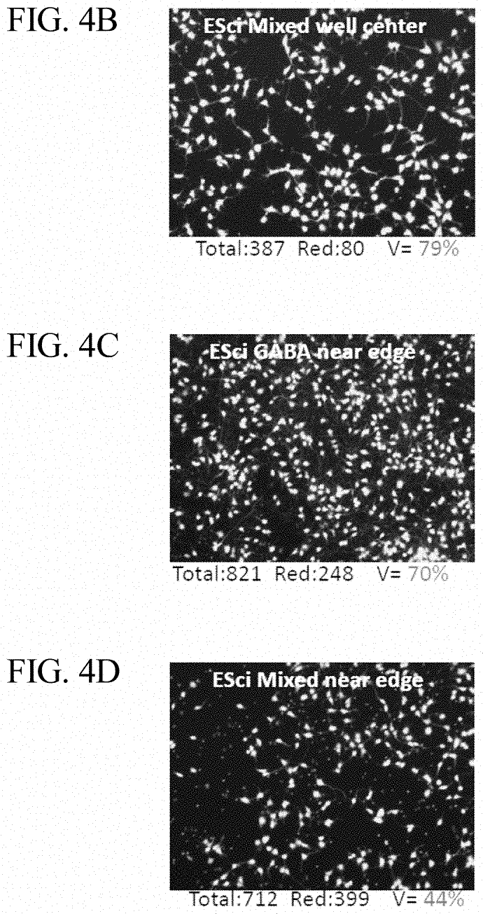

FIG. 3 is a schematic cross-sectional view illustrating a configuration of an inkjet head. As shown in FIG. 3, an inkjet head 300 has at least a liquid-holding part 320 which holds a cell suspension 310 containing cells C, a film-like member 330 on which a nozzle 331 is formed and which jets, from the nozzle 331, the cell suspension 310 held in the liquid-holding part as a liquid droplet 310′ by vibration, and an atmospheric opening part which opens an inside of the liquid-holding part 320 to the atmosphere. The inkjet head 300 preferably has a piezoelectric element 340.

In the inkjet head 300, a membrane can be deformed in a vertical direction by applying a voltage to the piezoelectric element 340 from a control device (not shown). Accordingly, the liquid droplet 310′ is formed while stirring the cell suspension 310 in the liquid-holding part 320, and thereby it is possible to inhibit nozzle clogging and to repeatedly form liquid droplets at high speed.

The above-described jetting step may be performed by operating a plurality of inkjet heads simultaneously or alternatively. For example, by causing the plurality of inkjet heads to each hold cell suspensions containing different cells, it is possible to manufacture a cell-containing container accommodating a plurality of types of cells.

### EXAMPLES

Next, the present invention will be described in more detail with reference to examples, but the present invention is not limited to the following examples.

### Experimental Example 1

(Examination of Survival Rate of Cells at Well Location)

iPS cells differentiation-induced into nerve cells were cultured on a 96-well plate, and survival rates at a center part and an edge portion of the wells were examined.

For cells, the following cells were used: cells (Elixirgen Scientific, Inc.) obtained by introducing a viral vector or mRNA expressing a transcription factor that induces differentiation into GABAergic nerve cells into iPS cells derived from a healthy subject, and cells (Elixirgen Scientific, Inc.) obtained by introducing a viral vector or mRNA expressing a transcription factor that induces differentiation into a nerve cell mixture (mixture of nerve cells) into iPS cells derived from a healthy subject.

A well surface of the 96-well plate is coated with a commercially available cell adhesive material (trade name, “iMatrix 511-silk,” manufactured by Nippi, Incorporated), and thereafter, each cell was seeded at 8×10⁴ cells/well (250 cells/mm²). Furthermore, iMatrix 511-silk is a cell adhesive material in which a human laminin fragment is expressed in silk moth. A commercially available serum-free medium (BrainPhys Neuronal Medium, STEMCELL Technologies Inc.) was used for a medium. In the present experimental example, cells adhered to the entire culture surface of the well.

Subsequently, each of the cells was cultured, and on the 8th day from the start of the culture, the cells were stained with Calcein-AM (DOJINDO LABORATORIES), Hoechst33342 (Thermo Fisher Scientific), and Ethidium Homodimer I (Thermo Fisher

Scientific), and observed with a fluorescence microscope. The cytoplasm of living cells was stained with Calcein-AM, the nuclei of both living cells and dead cells were stained with Hoechst33342, and the nuclei of dead cells were stained with Ethidium Homodimer I.

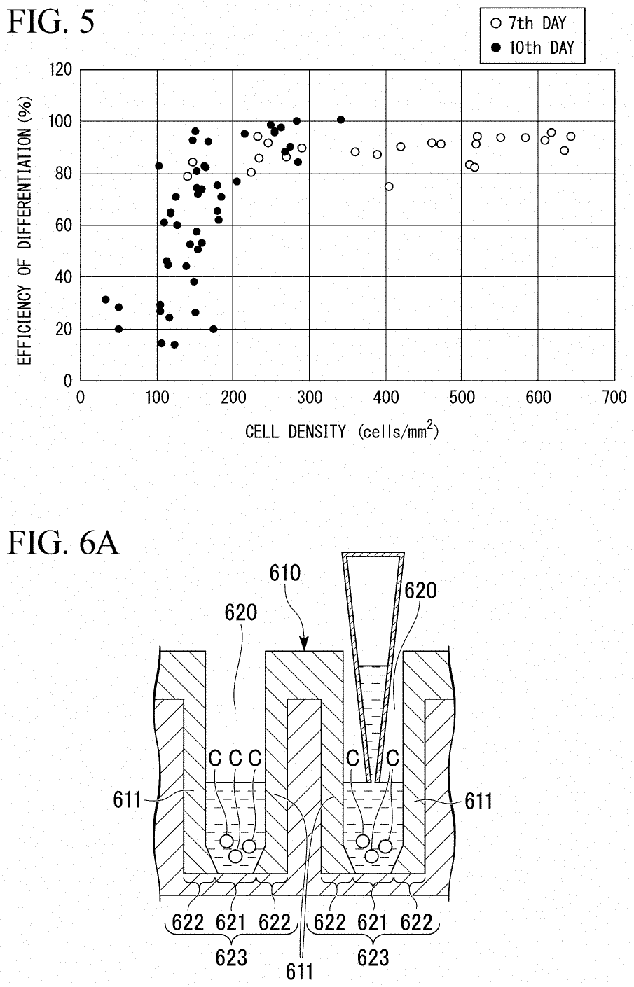

FIG. 4A is a fluorescence micrograph of GABAergic nerve cells in a center part of the well. FIG. 4B is a fluorescence micrograph of a nerve cell mixture in a center part of the well. FIG. 4C is a fluorescence micrograph of GABAergic nerve cells in an edge portion of the well. FIG. 4D is a fluorescence micrograph of a nerve cell mixture in an edge portion of the well. In FIG. 4A to FIG. 4D, “Total” represents the total number of cells, “Red” represents the number of dead cells, and “V” represents a survival rate.

As a result, a survival rate of the GABAergic nerve cells in the center part of the well was 89%. Furthermore, a survival rate of GABAergic nerve cells in the edge portion of the wells was 70%. Furthermore, a survival rate of the nerve cell mixture in the center part of the well was 79%. Furthermore, a survival rate of the nerve cell mixture in the edge portion of the well was 44%.

Based on the above results, it was clarified that a survival rate of cells decreased in the edge portion of the well when the cells adhered to the entire culture surface of the well.

### Experimental Example 2

(Examination of Cell Density and Efficiency of Differentiation)

iPS cells were seeded at various cell densities to be differentiation-induced into nerve cells, and efficiency of differentiation was examined. For the iPS cells, the following cells were used: cells (Elixirgen Scientific, Inc.) obtained by introducing a viral vector or mRNA expressing a transcription factor that induces differentiation into a nerve cell mixture (mixture of nerve cells) into iPS cells derived from a healthy subject.

The above-mentioned iPS cells were seeded on a 96-well plate at various cell densities. A commercially available serum-free medium (BrainPhys Neuronal Medium, STEMCELL Technologies Inc.) was used for a medium.

Subsequently, each of the cells was cultured and fixed with paraformaldehyde on the 7th or 10th day from the start of the culture. Subsequently, the cells were stained with βIII tubulin, which is a marker for nerve cells, by immunostaining, and observed with a fluorescence microscope to measure efficiency of differentiation into nerve cells.

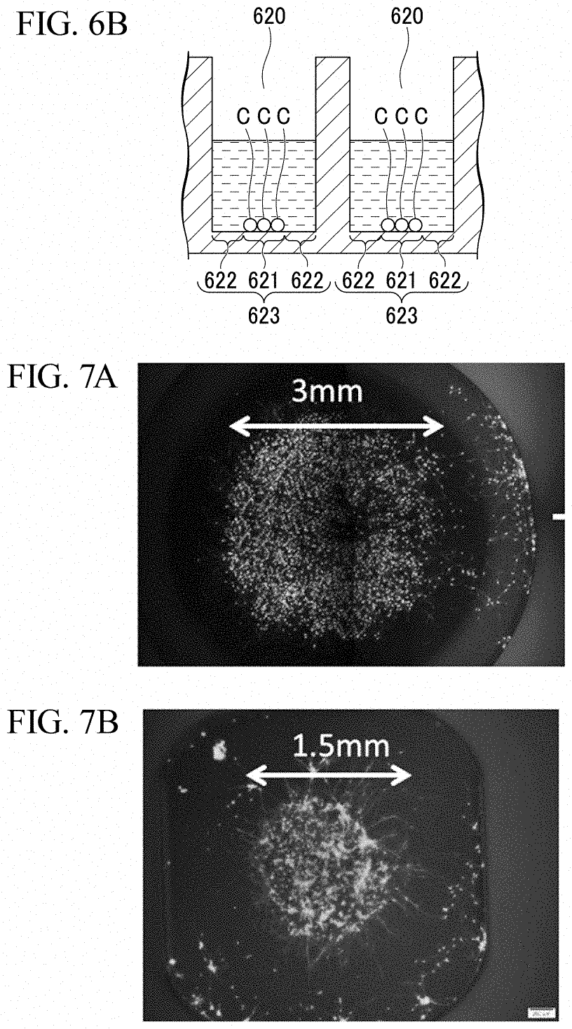

FIG. 5 is a graph showing a relationship between a cell density of iPS cells and efficiency of differentiation into nerve cells. FIG. 5 collectively shows results (indicated by white circles) of the cells fixed with paraformaldehyde on the 7th day from the start of culture, and results (indicated by black circles) of the cells fixed with paraformaldehyde on the 10th day from the start of culture. As a result, it was clarified that efficiency of differentiation into nerve cells showed a high value of 80% or more when iPS cells were seeded at a cell density of 220 cells/mm² or more.

### Experimental Example 3

(Examination of Method of Limiting Cell Adhesion Region in Well)

Based on the results of Experimental Examples 1 and 2, it was shown that cell death was inhibited and efficiency of differentiation was increased when iPS cells were seeded only in the center portion of the well at a cell density of 220 cells/mm² or more. Therefore, in the present experimental example, a method of seeding cells only in the center portion of the well was examined. Specifically, cells were seeded using a frame member covering an outer edge portion of the bottom surface of the well.

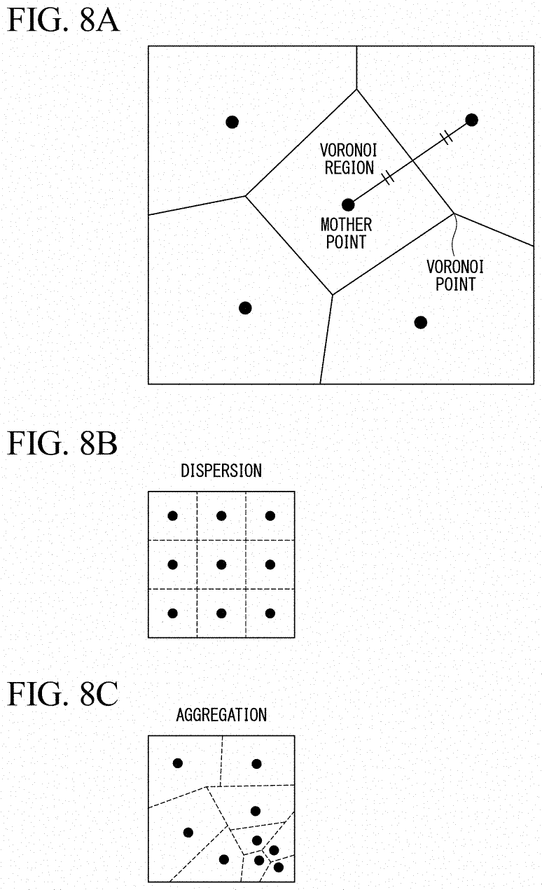

FIG. 6A and FIG. 6B are schematic cross-sectional views illustrating a frame member used in the present experimental example. As shown in FIG. 6A, a frame member 610 has a tubular part 611, and the tubular part 611 is used by being inserted into a well 620.

As shown in FIG. 6A, when the tubular part 611 is inserted into the well 620, an outer surface of a wall part of the tubular part 611 comes into contact with an inner wall of the well 620. As a result, in a bottom surface 623 of the well 620, an outer edge portion (second region 622) of the bottom surface 623 is covered with the wall part of the tubular part 611 by a thickness of the wall part of the tubular part 611. On the other hand, the center portion of the bottom surface 623 (first region 621, a lumen portion of the tubular part 611) is open.

Subsequently, as shown in FIG. 6A, when cells C are seeded in the lumen part of the tubular part 611, the cells adhere to the first region 621 but do not come into contact with the second region 622. Subsequently, as shown in FTG. 6B, when the frame member 610 is removed, the cells enter a state of adhering to the first region 621 at a high cell density, but not adhering to the second region 622. As will be described later, cells may actually adhere to the second region 622 in some cases, but the adhesion of cells to the second region 622 is not intended. Accordingly, a cell density in the second region 622 is much lower than a cell density in the first region 621.

In the present experimental example, nerve cells were seeded only in the center part of the well of the 96-well plate (well diameter of about 6 mm) using each of a frame member in which a diameter of a lumen part (diameter of an open portion at a bottom surface of a well) is about 3 mm, and a frame member in which a diameter of a lumen part is about 1.5 mm. Subsequently, the nerve cells were cultured, and on the 3rd day from the start of the culture, living cells were stained with Calcein-AM (DOJINDO LABORATORIES) and observed with a fluorescence microscope.

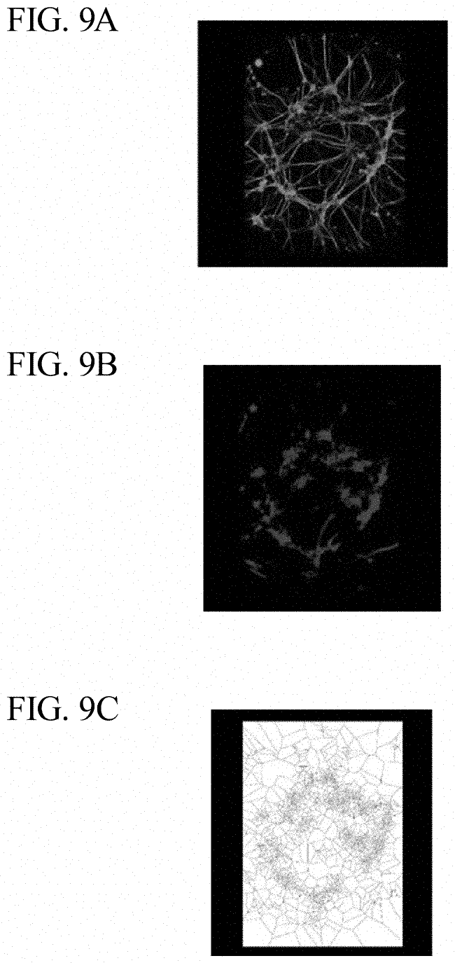

FIG. 7A and FIG. 7B are fluorescence micrographs. As a result, it was clarified that cells could adhere mainly to a vicinity of the center of the well regardless of which frame member was used.

This result indicates that a small number of cells can be seeded at a high cell density in a limited area of a well by the above-described method.

### Experimental Example 4

(Examination of Variation in Cell Adhesion)

Well surfaces of a 96-well plate were coated with a cell adhesive material at various concentrations. As the cell adhesive material, a commercially available cell adhesive material (trade name, iMatrix 511-silk, manufactured by Nippi, Incorporated) was used. Subsequently, iPS cells were seeded in the wells and cultured for 4 days. Thereafter, variation in cell adhesion was examined.

For the iPS cells, the following cells were used: cells (Elixirgen Scientific, Inc.) obtained by introducing a viral vector or mRNA expressing a transcription factor that induces differentiation into a nerve cell mixture (mixture of nerve cells) into iPS cells derived from a healthy subject.

In addition, variation in cell adhesion was evaluated by a degree of aggregation calculated by Formula (1).

Degree of aggregation (%)=A/B×100 (1)

[In Formula (1), A indicates a standard deviation of area of Voronoi regions of each cells, and B indicates an average value of areas of Voronoi regions of each cells, where a Voronoi region of a cell is a region surrounded by perpendicular bisectors in a case where a perpendicular bisector is drawn between a nucleus of a cell and a nucleus of a cell adjacent to the cell].

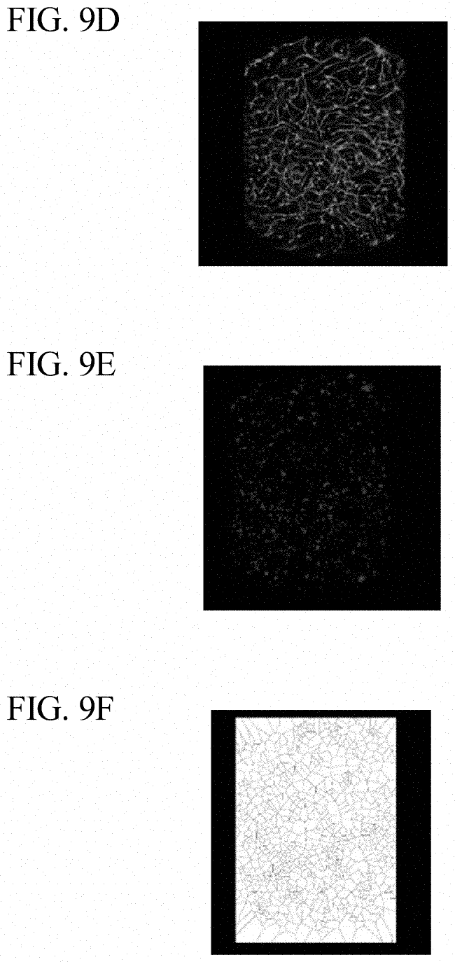

FIG. 8A to FIG. 8C are diagrams illustrating evaluation of variation in cell adhesion using Formula (1). FIG. 8A is a diagram illustrating a Voronoi diagram. The Voronoi diagram is used for particle dispersion evaluation and the like. As shown in FIG. 8A, in a Voronoi diagram, a perpendicular bisector of two mother points on the image is called a Voronoi boundary. In addition, a region surrounded by Voronoi boundaries created from each of mother points is called a Voronoi region. An image in which a Voronoi region is drawn from mother points is called a Voronoi diagram.

FIG. 8B is an example of a Voronoi diagram in a case where mother points are uniformly dispersed. Furthermore, FIG. 8C is an example of a Voronoi diagram in which mother points are unevenly dispersed (sometimes referred to as “aggregated” in the present specification). As shown in FIG. 8B and FIG. 8C, variation in areas of Voronoi regions occurs depending on degrees of dispersion of mother points. That is, a degree of dispersion of mother points can be evaluated by evaluating variation in Voronoi regions in the image.

In the present experimental example, a Voronoi diagram was applied to cultured cells to evaluate variation in cell adhesion. Specifically, first, cultured cells were immunostained with an antibody against tubulin, which is a marker for nerve cells, and the nuclei of the cells were stained with 4′,6-diamidino-2-phenylindole (DAPI).

Subsequently, the cells were observed with a fluorescence microscope to create a Voronoi diagram with the cell nuclei in the fluorescence microscope image as mother points. Subsequently, an area of each of Voronoi regions in the Voronoi diagram was calculated. Subsequently, a CV value of the calculated area of each of the Voronoi regions was defined as a degree of aggregation (%) in Formula (1), and a degree of aggregation was calculated.

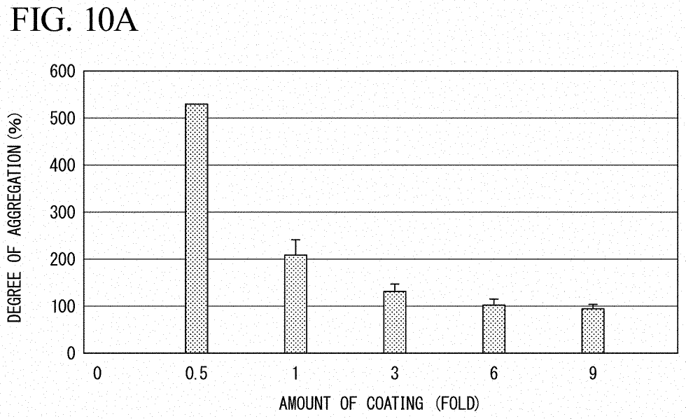

FIG. 9A to FIG. 9F are fluorescence micrographs and Voronoi diagrams each showing a typical result of calculating a degree of aggregation of cells. FIG. 9A to FIG. 9C show results of iPS cells cultured in a well coated with a commercially available cell adhesive material (trade name, “iMatrix 511-silk,” manufactured by Nippi, Incorporated) at a 1-fold concentration (4.7×10⁻¹² g/min²) as a cell adhesive material. FIG. 9A is a fluorescence micrograph in which an immunostaining image using an anti-βIII tubulin antibody and a staining image by DAPI are superimposed. FIG. 9B is a fluorescence micrograph showing nuclei of cells stained with DAPI. FIG. 9C is a Voronoi diagram created with the nuclei of cells of FIG. 9B as mother points. Based on FIG. 9C, a degree of aggregation calculated by Formula (1) was 252%.

FIG. 9D to FIG. 9F show results of iPS cells cultured in a well coated with a commercially available cell adhesive material (trade name, “iMatrix 511-silk,” manufactured by Nippi, Incorporated) at a 9-fold concentration (4.23×10⁻¹¹ g/mm²) as a cell adhesive material. FIG. 9D is a fluorescence micrograph in which an immunostaining image using an anti-βIII tubulin antibody and a staining image by DAPI are superimposed. FIG. 9E is a fluorescence micrograph showing nuclei of cells stained with DAPI. FIG. 9F is a Voronoi diagram created with the nuclei of cells of FIG. 9E as mother points. Based on FIG. 9F, a degree of aggregation calculated by Formula (1) was 88%.

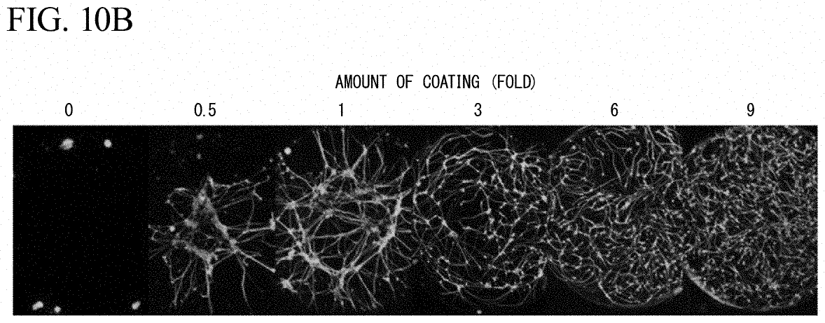

FIG. 10A is a graph showing a degree of aggregation calculated by culturing iPS cells in wells coated with a commercially available cell adhesive material (trade name, “iMatrix 511-silk,” manufactured by Nippi, Incorporated) as a cell adhesive material at a 0-fold concentration (no coating), at a 0.5-fold concentration (2.4×10⁻¹² g/mm²), at a 1-fold concentration (4.7×10⁻¹² g/mm²), at a 3-fold concentration (1.4×10⁻¹¹ g/mm²), at a 6-fold concentration (2.8×10⁻¹¹ g/mm²), and at a 9-fold concentration (4.23×10⁻¹ g/mm²), and thereby creating Voronoi diagrams.

Furthermore, FIG. 10B is a fluorescence micrograph which superimposes an immunostaining image using an anti-βIII tubulin antibody and a staining image by DAPI which were taken in the same manner as above after culturing iPS cells in a well coated with a commercially available cell adhesive material (trade name, iMatrix manufactured by Nippi, Incorporated) at each concentration.

As a result, the cells were peeled off at a 0-fold concentration, and thereby a Voronoi diagram could not be created, and an aggregation rate could not be calculated. It was also clarified that an aggregation rate of cells tended to decrease when a concentration of the cell adhesive material was increased. Furthermore, it was clarified that a degree of decrease in an aggregation rate became gradual when a concentration of the cell adhesive material was 3-fold or 6-fold. This result shows that a 3-fold or 6-fold concentration of the cell adhesive material is a sufficient amount.

Based on the above results, it was clarified that, by applying a Voronoi diagram to cultured cells, a cultured state can be quantitatively measured using a value representing a degree of aggregation of cells.

### Experimental Example 5

(Examination 1 Using MEA Plate)

Nerve cells and astrocytes were seeded in wells of an MEA plate. Using the same frame member as that used in Experimental Example 3, cells obtained by mixing nerve cells (manufactured by Elixirgen Scientific, Inc.) and astrocytes (manufactured by Elixirgen Scientific, Inc.) were seeded on wells of the MEA plate (product name “CytoView MEA 48,” Axion Biosystems). An area of a lumen part of the frame member used (area of an open portion on a bottom surface of the well) was 22 mm².

A cell density of the seeded cells was 6,400 cells/mm² for the nerve cells and 1,600 cells/mm² for the astrocytes. Furthermore, wells in which cells were seeded without using a frame member were also prepared for comparison.

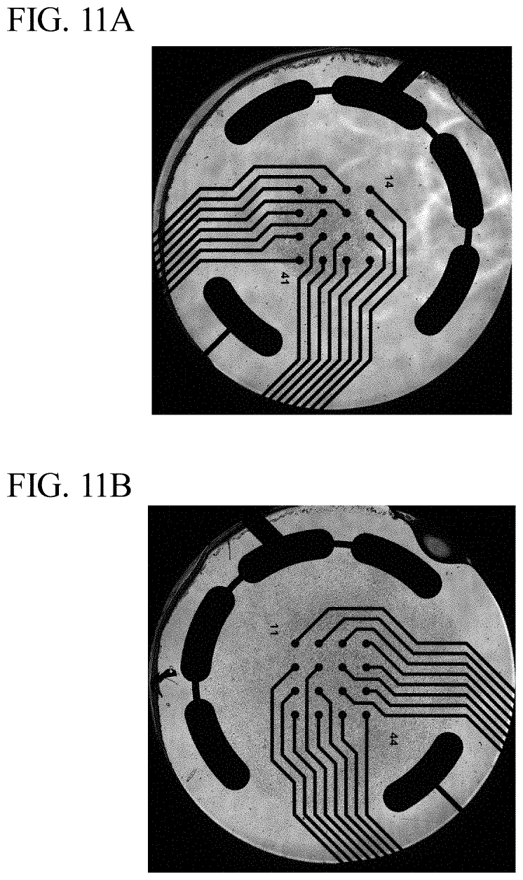

FIG. 11A is a photograph showing a result of seeding cells only in a center part of a well using a frame member. FIG. 11B is a photograph showing a result of seeding cells in a well without using a frame member. As a result, it was clarified that the cells adhered to the bottom surface of the well without any problem even when the cells were seeded only in the center part of the well using the frame member.

Furthermore, as will be described later, it was clarified that an extracellular potential could be measured even when the cells were seeded only in the center part of the well of the MEA plate in which the electrode array was present.

### Experimental Example 6

(Examination 2 Using MEA Plate)

Using a frame member in which an area of a lumen part (area of an open portion on a bottom surface of a well) was about 1.8 mm², and a frame member in which an area of a lumen part was 3.1 mm², cells obtained by mixing nerve cells (manufactured by Elixirgen Scientific, Inc.) and astrocytes (manufactured by Elixirgen Scientific, Inc.) were seeded on wells of an MEA plate (product name “CytoView MEA 48,” Axion Biosystems) in the same manner as in Experimental Example 5 except that the number of cells seeded was changed. A ratio of the number of nerve cells to the number of astrocytes was defined as the number of nerve cells:the number of astrocytes=about 4:1.

The cells were seeded and cultured for 14 days, and thereafter, the number of s (spikes) of nerve cells per 10 minutes in each well was measured.

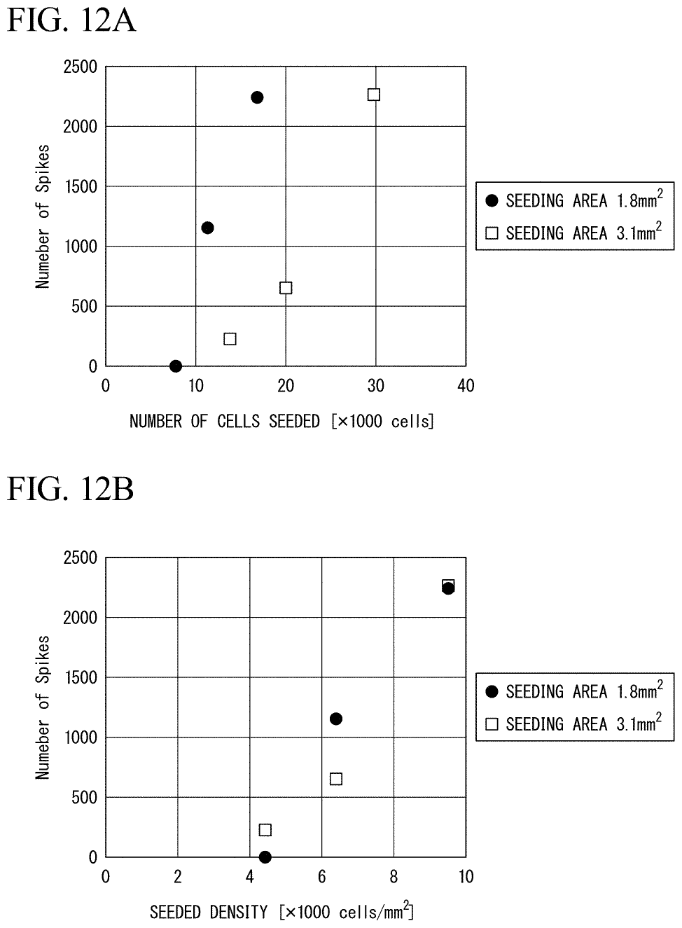

FIG. 12A and FIG. 12B are graphs showing measurement results of the number of spikes. A horizontal axis of FIG. 12A is the number of nerve cells seeded per well, and a vertical axis is the number of spikes. A horizontal axis of FIG. 12B is a seeding density of nerve cells in each well, and a vertical axis is the number of spikes.

Based on the results of FIG. 12A, it was clarified that, when the number of cells seeded per well was the same, the number of spikes per unit time was larger in the case of using the frame member in which the area of the lumen part was 1.8 mm², which was a small seeding area (high seeded cell density), as compared to the case of using the frame member in which the area of the lumen part was about 3.1 mm².

Furthermore, based on the result of FIG. 12B, it was clarified that the seeded cell density was dominant in the correlation with the number of spikes, and even when the number of cells seeded is different, the same number of spikes can be generated as long as the seeded cell density is the same.

Based on these results, it was clarified that, in a case where the number of nerve cells seeded on the MEA plate is constant, by limiting a region in which a seeded cell density is high to an electrode portion, it is possible to increase the number of wells while maintaining functions and to reduce a cost for cells per well.

### Experimental Example 7

(Examination 3 Using MEA Plate)

Nerve cells and astrocytes were seeded in wells of an MEA plate, and their responsiveness to a drug was evaluated.

In the same manner as in Experimental Example 3, cells obtained by mixing nerve cells (manufactured by Elixirgen Scientific, Inc.) and astrocytes (manufactured by Elixirgen Scientific, Inc.) were seeded on wells of the MEA plate (product name “CytoView MEA 48,” Axion Biosystems). An area of a lumen part of the frame member used (area of an open portion on a bottom surface of the well) was 22 mm². A cell density of the seeded cells was 6,400 cells/mm² for the nerve cells and 1,600 cells/mm² for the astrocytes.

Subsequently, the cells were seeded and cultured for 49 days. Thereafter, 4-Aminopyridine (4-AP), which is an Na channel blocker, and Picrotoxin, which is a GABA antagonist, were each added into the wells of the MEA plate, and a potential fluctuation was measured for 10 minutes. A concentration of each of the drugs added was changed in several stages. Actions of 4-AP and Picrotoxin are both in an excitement direction.

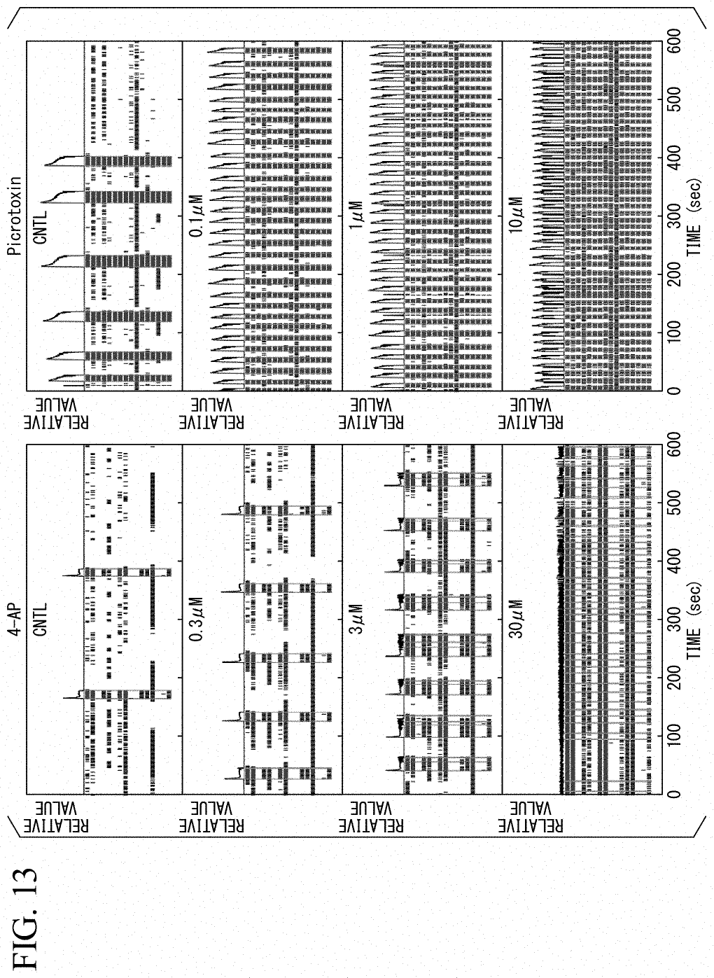

FIG. 13 is a raster plot showing measurement results. In FIG. 13, a vertical axis at the top of each graph indicates the integrated number of spikes. The bottom of each graph shows spikes detected at each of the 16 electrodes per well. Furthermore, “CTRL” indicates the measurement result of control wells into which a drug was not added.

As a result, an increase in spike frequency was recognized regardless of which drug was added. It was also clarified that a spike frequency increased in a drug concentration-dependent manner. Based on these results, it was clarified that drug evaluation can be performed while reducing the number of nerve cells used by seeding the nerve cells only in the center part of the well of the MEA plate.

The present invention includes the following aspects.

[1] A cell-containing container including at least one recessed part accommodating cells, in which the cells adhere to a bottom surface of the recessed part, and a cell density in a first region of the bottom surface is 250 to 20,000 cells/mm², and a cell density in a second region surrounding a periphery of the first region is less than 250 cells/mm².

[2] The cell-containing container according to [1], in which a cell density in the first region is 500 to 10,000 cells/mm².

[3] The cell-containing container according to [1] or [2], in which the cells are primary cells, stem cells or induced pluripotent stem (iPS) cells, in which the cells are derived from a patient or a healthy subject, or cells differentiated from the iPS cells.

[4] The cell-containing container according to any one of [1] to [3], including a plurality of recessed parts, in which each of the recessed parts accommodates cells derived from the same patient or the same healthy subject, and the plurality of recessed parts include recessed parts each accommodating cells derived from different patients or different healthy subjects.

[5] The cell-containing container according to any one of [1] to [4], in which a degree of aggregation of cells in the first region is 100% or less, which is calculated by Formula (1):

Degree of aggregation (%)=A/B×100 (1)

[In Formula (1), A indicates a standard deviation of area of Voronoi regions of each cells, and B indicates an average value of areas of Voronoi regions of each cells, where a Voronoi region of a cell is a region surrounded by perpendicular bisectors in a case where a perpendicular bisector is drawn between a nucleus of a cell and a nucleus of a cell adjacent to the cell].

[6] The cell-containing container according to any one of [1] to [5], in which the recessed part accommodates (i) nerve cells, and (ii) at least one cells selected from the group consisting of glial cells, vascular endothelial cells, and smooth muscle cells.

[7] The cell-containing container according to any one of [1] to [6], in which the recessed part accommodates nerve cells, and an electrode array is provided on the bottom surface of the recessed part.

[8] A method for evaluating a test substance, the method including a step of adding a test substance into a recessed part of a cell-containing container which has at least one recessed part containing cells and in which a cell density in a center of a bottom surface of the recessed part is higher than that in an edge portion of the bottom surface of the recessed part; and a step of detecting spontaneous firing of nerve cells through an electrode array provided on the bottom surface of the recessed part.

[9] A method for manufacturing a cell-containing container, the method including a step of controlling the number and positions of cells and jetting the cells by an inkjet technique, where the controlling is performed such that, in a recessed part of a container having at least one recessed part, a cell density in a center of a bottom surface of the recessed part is 250 to 20,000 cells/mm², and a cell density in an edge portion of the bottom surface of the recessed part is less than 250 cells/mm².

[10] The method for manufacturing a cell-containing container according to [9], in which the step of jetting the cells is performed by an inkjet head including at least a liquid-holding part which holds a cell suspension containing cells, a film-like member on which a nozzle is formed and which jets, from the nozzle, the cell suspension held in the liquid-holding part as liquid droplets by vibration, and an atmospheric opening part which opens an inside of the liquid-holding part to the atmosphere.

[11] The method for manufacturing a cell-containing container according to [10], in which the step of jetting the cells is performed by operating a plurality of inkjet heads simultaneously or alternatively.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

100, 200 Cell-containing container

110, 110a, 110b, 110c, 110d, 110e, 110f, 620 Recessed part (well)

120, 623 Bottom surface

121, 621 First region

122, 622 Second region

300 Inkjet head

310 Cell suspension

310′ Liquid droplet

320 Liquid-holding part

330 Film-like member

331 Nozzle

340 Piezoelectric element

610 Frame member

611 Tubular part

C, C¹, C², C³, C⁴, C⁵ Cell

### Patent Documents

[Patent Document 1] Japanese Patent No. 5607535

[Patent Document 2] Published Japanese Translation No. 2015-510401 of the PCT International Publication

What is claimed is:

1. A cell-containing container, comprising:
at least one recessed part accommodating cells,
wherein the cells adhere to a bottom surface of the recessed part, and
a cell density in a first region of the bottom surface is 250 to 20,000 cells/mm², and a cell density in a second region surrounding a periphery of the first region is less than 250 cells/mm².

2. The cell-containing container according to claim 1, wherein a cell density in the first region is 500 to 10,000 cells/mm².

3. The cell-containing container according to claim 1, wherein the cells are primary cells, stem cells or induced pluripotent stem (iPS) cells, wherein the cells are derived from a patient or a healthy subject, or cells differentiated from the iPS cells.

4. The cell-containing container according to claim 1, comprising:
a plurality of recessed parts,
wherein each of the recessed parts accommodates cells derived from the same patient or the same healthy subject, and
the plurality of recessed parts include recessed parts each accommodating cells derived from different patients or different healthy subjects.

5. The cell-containing container according to claim 1, wherein a degree of aggregation of cells in the first region is 100% or less, which is calculated by Formula (1):
degree of aggregation (%)=A/B×100 (1)
[in Formula (1), A indicates a standard deviation of area of Voronoi regions of each cells, and B indicates an average value of areas of Voronoi regions of each cells, where a Voronoi region of a cell is a region surrounded by perpendicular bisectors in a case where a perpendicular bisector is drawn between a nucleus of a cell and a nucleus of a cell adjacent to the cell].

6. The cell-containing container according to claim 1, wherein the recessed part accommodates (i) nerve cells, and (ii) at least one cells selected from the group consisting of glial cells, vascular endothelial cells, and smooth muscle cells.

7. The cell-containing container according to claim 1, wherein the recessed part accommodates nerve cells, and an electrode array is provided on the bottom surface of the recessed part.

8. A method for evaluating a test substance, the method comprising:
adding a test substance into a recessed part of a cell-containing container which has at least one recessed part containing cells and in which a cell density in a center of a bottom surface of the recessed part is higher than that in an edge portion of the bottom surface of the recessed part; and
detecting spontaneous firing of nerve cells through an electrode array provided on the bottom surface of the recessed part.

9. A method for manufacturing a cell-containing container, the method comprising controlling the number and positions of cells and jetting the cells by an inkjet technique, where the controlling is performed such that, in a recessed part of a container having at least one recessed part, a cell density in a center of a bottom surface of the recessed part is 250 to 20,000 cells/mm², and a cell density in an edge portion of the bottom surface of the recessed part is less than 250 cells/mm².

10. The method for manufacturing a cell-containing container according to claim 9, wherein jetting the cells is performed by an inkjet head including at least a liquid-holding part which holds a cell suspension containing cells, a film-like member on which a nozzle is formed and which jets, from the nozzle, the cell suspension held in the liquid-holding part as liquid droplets by vibration, and an atmospheric opening part which opens an inside of the liquid-holding part to the atmosphere.

11. The method for manufacturing a cell-containing container according to claim 10, wherein jetting the cells is performed by operating a plurality of inkjet heads simultaneously or alternatively.
